(19) United States
(12) Patent Application Publication
(10) Pub. No.: US 2026/0102104 A1
(43) Pub. Date: Apr. 16, 2026

(54) Methods and systems for determining a state of working memory of a mammalian brain

(71) Applicant: Stephanie Littell, Lexington, MA (US)

(72) Inventor: Stephanie Littell, Lexington, MA (US)

(21) Appl. No.: 19/358,967

(22) Filed: Oct. 15, 2025

Related U.S. Application Data
(60) Provisional application No. 63/708,166, filed on Oct. 16, 2024.

(51) Int. Cl. A61B 5/374; A61B 5/00; A61B 5/378

(52) U.S. Cl. CPC A61B 5/374; A61B 5/378; A61B 5/6814; A61B 5/742

(57) ABSTRACT

Methods and systems for determining a state of working memory of a mammalian brain are disclosed. An example method includes positioning a device on an individual and obtaining measurement data of the individual's brain. The measurement data includes time series data collected from sensors over a period of time including at least one stimulus event. Temporal information of the stimulus event is obtained. A spectrogram of the measurement data is computed for each of the sensors. A classifying technique is used to identify onsets of activation and durations of activation within at least one frequency sub-band of the spectrogram in time. A state of working memory is determined based on the onsets and durations identified. The state of working memory temporally correlated with the stimulus event is output. Methods and systems as disclosed may be useful for quantitatively or qualitatively evaluating cognitive performance of a mammalian brain.

### RELATED APPLICATION

This application claims the benefit of U.S. Provisional Application No. 63/708,166, filed on Oct. 16, 2024. The entire teachings of the above application are incorporated herein by reference.

### BACKGROUND

Working memory in the mammalian brain comprises a form of short-term memory that may retain limited amounts of information for purposes including, as non-limiting examples, cognitive processing or complex task execution. Furthermore, measurements associated with working memory, e.g., using neuroimaging modalities such as electroencephalography (EEG), may be capable of identifying states of working memory of a brain, for example, an aroused or active processing state versus an idle state, or changes thereof. As such, the ability to evaluate working memory may be useful for a variety of applications including, but not limited to, determining efficacy of education in a classroom environment or tracking the function or health of a brain over time.

### SUMMARY

Evaluating working memory of a mammalian brain in different environments, for example, in an at-home, workplace, or classroom setting, may be useful for providing information regarding a state or changes thereof of the working memory of an individual. In particular, quantitative and/or longitudinal measurements of working memory may reveal dynamic changes in the state of working memory as a result or time, environment, stimulus, or other factors. Portable systems and devices, including portable processing devices, e.g., a mobile phone or laptop, and neuromonitoring systems, e.g., electroencephalography (EEG) devices, may further provide accessible tools for evaluating the working memory of individuals. Methods and systems for determining a state of working memory of a mammalian brain are disclosed herein.

In an example embodiment, a method for determining a state of working memory of a mammalian brain includes positioning a measurement device on an individual at an anatomical region to be studied and obtaining measurement data of the individual's brain from the measurement device. The measurement data includes time series data collected from at least one sensor of the measurement device over a period of time, wherein the period of time includes at least one stimulus event. The method further includes obtaining temporal information of a start time, an end time, or both a start time and an end time corresponding to the at least one stimulus event and computing a spectrogram of the time series data of the measurement data collected from the at least one sensor. The method further includes identifying using a classification technique of any number of onsets of activation and any number of durations of activation within at least one frequency sub-band of the spectrogram in time, wherein an onset of the any number of onsets comprises a time point in the spectrogram with an amplitude exceeding a threshold value, and a duration of the any number of durations comprises a length of time in the spectrogram in which the amplitude remains above the threshold value. The method further includes determining a state of working memory of the brain in the period of time based on the any number of onsets identified and the any number of durations identified of the at least one frequency sub-band of the spectrogram and outputting the state of working memory. The state of working memory is temporally correlated with the at least one stimulus event using the temporal information.

The measurement data may include recordings of electrical activity, e.g., electrical activity associated with electrical activity of the individual's brain. The measurement data may be recorded using sensors configured to record electrical activity, e.g., electroencephalography sensors. The measurement data can be recorded and processed asynchronously, e.g., the measurement data may be saved for processing at a later time or in conjunction with later acquired data.

The state of working memory may include task invocation, task execution, memory encoding, or memory decoding.

Computing the spectrogram may include applying a frequency-based decomposition method. Computing the spectrogram may further include applying one or more data preprocessing techniques to the measurement data. The spectrogram may be computed over a period of time, e.g., the frequency-based decomposition of a discrete time signal may be applied to successive time intervals of the discrete time signal.

The at least one frequency sub-band may include alpha waves, beta waves, gamma waves, theta waves, delta waves, or some combination thereof or therebetween.

The classification technique may include a machine learning method, a principal component analysis, a support vector machine, a Bayesian inference, a Recurrent Neural Network, or a combination thereof.

The anatomical region may include a forehead of the individual. The anatomical region may also include a pre-frontal cortex or a frontal lobe. The measurement device may further be positioned at one or more anatomical regions concurrently to acquire measurements at the one or more anatomical regions at the same time. The measurement device can also be positioned at the one or more anatomical locations sequentially.

The at least one stimulus event may include a working memory task, a cognitive task, or a motor task. For example, a working memory task may include a recall task, a cognitive task may include a mental calculation task, and a motor task may include handwriting a signature.

The method may further include determining a health status of the working memory of the individual based on the state of working memory and one or more of the any number of onsets of the at least one sub-band, the any number of durations of the at least one sub-band, and the temporal information corresponding with the at least one stimulus event. The temporal information may include the start time, the end time, or the start time and the end time of the at least one stimulus event.

In another example embodiment, a system for determining a state of working memory of a mammalian brain includes a measurement device comprising at least one sensor, the measurement device configured to be positioned on an individual at an anatomical region to be studied. The system further includes a computer-based device configured to connect with the measurement device. The computer-based device is further configured to obtain measurement data of the individual's brain from the measurement device, wherein the measurement data comprise time series data collected from the at least one sensor over a period of time that includes at least one stimulus event. The computer-based device is further configured to obtain temporal information of a start time, an end time, or both a start time and an end time corresponding to the at least one stimulus event, and to compute a spectrogram of the time series data of the measurement data collected from the at least one sensor. The computer-based device is further configured to identify using a classification technique any number of onsets of activation and any number of durations of activation within at least one frequency sub-band of the spectrogram in time. An onset of the any number of onsets includes a time point in the spectrogram with an amplitude exceeding a threshold value and a duration of the any number of durations includes a length of time in the spectrogram in which the amplitude remains above the threshold value. The computer-based device is further configured to determine a state of working memory of the brain in the period of time based on the any number of onsets identified and the any number of durations identified of the at least one frequency sub-band of the spectrogram. The computer-based device is further configured to output the state of working memory, the state of working memory temporally correlated with the at least one stimulus event using the temporal information. Temporally correlated can include, for example, that a stimulus event occurs and then working memory adjusts accordingly in response.

The at least one sensor may be configured to measure electrical activity. The at least one sensor may include an electrode, for example, in an EEG device.

The state of working memory determined by the computer-based device may include task invocation, task execution, memory encoding, or memory decoding.

The computer-based device may be configured to compute the spectrogram using a frequency-based decomposition method. The computer-based device may be further configured to compute the spectrogram by applying one or more pre-processing techniques.

The computer-based device may be configured to apply as a classification technique a machine learning method, a principal component analysis, a support vector machine, a Bayesian inference, a Recurrent Neural Network or a combination thereof.

The computer-based device may include a cell phone, a tablet, a computer, or a laptop computer.

The computer-based device may be configured to provide the stimulus event via a graphical interface. The system may further configure the graphical interface. The computer-based device may be configured to output a representation of the state of working memory via the graphical interface.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic diagram of an example embodiment of a system for measuring a head and/or face area of an individual.

FIG. 2 is a flow diagram of an example embodiment of a method of the present invention.

FIG. 3 is a schematic diagram of an example embodiment of a system for evaluating a state of working memory of a user's brain with sensors positioned on the head of the user.

FIGS. 4A and 4B illustrate example graphical interfaces of task execution by a user, which may be rendered on a computer-based device. Such graphical interfaces may be employed by a system for determining a state of working memory of the brain of the user, according to an example embodiment.

FIG. 5 is a schematic diagram of a computer network in which the present invention may be implemented.

FIG. 6 is a block diagram of a computer node in the network of FIG. 5.

FIG. 7 is a schematic illustration of a plot of time series data of brain activity acquired using a sensor, according to an example embodiment.

FIG. 8 illustrates dividing time series data into discrete time bins, according to an example embodiment.

FIG. 9 is a schematic illustration of plots of frequency components of the discrete time bins of FIG. 8.

FIG. 10 illustrates an example method of finding a starting time and an ending time for a frequency entrainment evaluated in time series data, according to an example embodiment.

FIG. 11 illustrates another example method of finding a starting time and an ending time for a frequency entrainment evaluated in time series date, according to an example embodiment.

FIG. 12 illustrates an example backwards processing of time series data to identify verify associated start and end times of a selected entrainment frequency, according to an example embodiment.

### DETAILED DESCRIPTION

A description of example embodiments follows.

Methods and systems for determining a state of working memory of a brain are described. Embodiments are useful for identifying the state of working memory of the mammalian brain at a point in time and may be useful in making decisions regarding working memory, for example, working memory health, learning efficacy, and other mental or cognitive traits.

Working memory in the mammalian brain comprises a short-term memory that is useful for retaining small amounts of information, including sensory information as a non-limiting example, in a readily accessible form. The small amounts of information retained within working memory may be used in a number of ways, including recalling or performing a mental or cognitive operation as non-limiting examples.

Neurons act and communicate through electrical signaling. When stimulated via input, a neuron may be stimulated enough to achieve an action potential, such that an electrical impulse or firing occurs. As countless numbers of neurons are firing at any time and thus propagating signaling, the resulting nearby electrical field is evolving as well. This bursting, or spiking, may be used to determine a state of working memory.

Functional neuroimaging techniques, e.g., electroencephalography (EEG) and magnetoencephalography (MEG), may enable non-invasive monitoring of neural activation, including working memory. Patterns in local field potentials (LFP) and spiking, or bursting, of electrical activity from the pre-frontal cortex of monkeys performing a working memory task, as recorded using EEG, are described in the article titled “Gamma and beta bursts underlie working memory” by Lundqvist et al., Neuron, 2016, 90 (1):152-164, the teachings of which are incorporated by references in their entirety. Lindqvist describes brief bursts of narrow-band gamma oscillations, varied in time and frequency, accompanying encoding and re-activation of sensory information. Beta oscillations also occurred in brief, variable bursts but reflected a default state interrupted by encoding and decoding.

In the context of a task including working memory, for example, a cognitive task, a mental puzzle, a word puzzle, an auditory task, and others, the onset and duration of bursting of beta waves, the onset and duration of bursting of gamma waves, or some combination thereof may reveal the state of working memory, including task invocation, task execution, and timings thereof. For example, a new auditory task may give rise to progressively entrained beta spikes that lock phase because they are firing at a periodic frequency for some time and may form the carrier frequency of the gamma frequency spiking that will ensue for task execution. Further, in tasks including a sequence of stimulus events, a subsequent task may result in additional bursting of beta waves.

In the prefrontal cortex, bursting entrainment in the beta (or other) frequency range is anti-correlated with bursting in the gamma frequency range. Before bursting in the gamma frequency range takes place, there may be bursting in the beta frequency range, which may be indicative of task control. Task control may infer inhibition of the previous task via prefrontal pyramidal neurons, possibly orienting towards a new task rule. Such a signal may carry its own center frequency and sideband frequencies and may be used to mark an earlier start time for a current task if task switching is included as well.

As inhibition may reduce excitation amplitude or prevent threshold firing altogether, the presence of bursting in the beta frequency range may reflect a new task rule rather than continued execution of a previous task. Inhibition requires hyperpolarization of a neuron. Inhibitory postsynaptic potentials (IPSPs) take place when negative ions such as Cl− influx to the postsynaptic cell, or when positive ions such as K+ efflux. The reversal potential of an IPSP can drive the neuron membrane to 0 mV from for example resting potential of −60 mV.

Further, when performing the task, information may need to be encoded into memory or decoded from memory. Encoding and decoding may also constitute activations that occur at times independent from task invocation or task execution. The beta frequency wave, as the carrier frequency, carries the modulating gamma frequencies which may correlate with specific tasks. Task information may be carried to regions of the brain using the beta carrier frequency wave. As the beta carrier frequency wave is frequency modulated, the information may be conveyed without significant interference from other carrier waves and may be measured. These measurements may be used to determine further states of working memory of a brain, for example, task invocation, task execution, encoding, or decoding.

This may be tested for an individual by first setting up a task according to a first task rule and measuring beta response, then supplying a different task rule and measuring (beta) center frequency shift. A neuronal difference in voltage across the membrane is necessary for spiking to occur. Subthreshold and threshold current flow takes place in neuronal circuits. (>1 mV/mm voltage gradient). Voltage and current are related through the electrical equation V=RI. If current I were to drop in a circuit, for example, as in cardiac output associated with aging, to maintain voltage V at similar levels, resistance R would necessarily increase. Although resistance should not affect an idealized FM system directly in terms of beta center frequency and modulation, it can affect a sharpness of the frequency response or select-frequency amplification. In an effective neuronal capacitor network, increasing the resistance can decrease a phase shift at a given frequency. The higher resistance can slow down charging and discharging of capacitors, so it can take longer for any entrainment towards a center frequency to manifest.

As cardiac output drops 1% per year from peak, it may follow that fluidic cardiovascular current drops by that amount annually. Corresponding transduction of energy, nutrients and pulsation may decrease brain current flow through the blood-brain barrier in such a closed system (where no delivery occurs from the outside). A larger percentage may occur (independent of draw demand) as rotational fluidic component may be more than half of delivery. With regards to transmittal of energy, rotational delivery includes an angular squared component term due to torque, multiplied by a radius-from-center squared term. This contrasts with simpler “forward” translational fluid movement which includes only a squared velocity term. Both can be roughly involving an equivalent amount of fluidic mass, so that the mass term is the same, i.e., not contributing any energy difference between the two different motions.

As neurons can be treated at their synaptic clefts as an effective capacitor, charging and discharging, they can be modeled in voltage terms as

$${V}_{c} ( t ) = {V}_{source} ( 1 - {e}^{- t / RC} ) .$$

In the above equation, V_{c}(t) is the voltage of the effective capacitor over time, V_{source} is a source voltage, t represents time, R represents resistance of the capacitor, and C represents capacitance of the capacitor. Collectively the neurons could be modeled to function as an aggregate capacitor circuit. Seeing how effective Resistance R and effective current I may change over years of aging, modeling such changes can be useful as it can showcase processing delays and trouble with timely memory retrieval. The voltage required to depolarize or hyperpolarize can be general over time, e.g., to charge or discharge an effective capacitor. Over a longer sampling interval, such as from one year to the next year, a required capacitor voltage can be analogous. Thus,

$${V}_{{c}_{old}} ( T ) = {V}_{{c}_{new}} ( T )$$

$${V}_{{source}_{old}} ( 1 - {e}^{- {t}_{old} / {R}_{old} C} ) = {V}_{{source}_{new}} ( 1 - {e}^{- {t}_{new} / {R}_{new} C} )$$

wherein “old” denotes parameters at a prior or older time point and “new” denotes parameters of at a more recent time point. If values of V_{source} and C are presumed to be equivalent over time as activating voltage requirements are still in physical force as are synaptic cleft charge properties, then approximately

$$1 - {e}^{- {t}_{old} / {R}_{old} C} = 1 - {e}^{- {t}_{new} / {R}_{new} C} .$$

It may then follow that:

$${e}^{- {t}_{old} / {R}_{o l d} C} = {e}^{- {t}_{new} / {R}_{new} C}$$

$$- {t}_{new} / {R}_{new} C ln ( e ) = - {t}_{old} / {R}_{old} C ln ( e )$$

$${t}_{new} / {R}_{new} C = {t}_{old} / {R}_{old} C$$

$${R}_{new} / {R}_{old} = {r}_{new} / {t}_{old}$$

This ratio of Resistance increases over time, e.g., as an individual ages, can be imputed by the increase in time to achieve a center frequency for the same task, in other words.

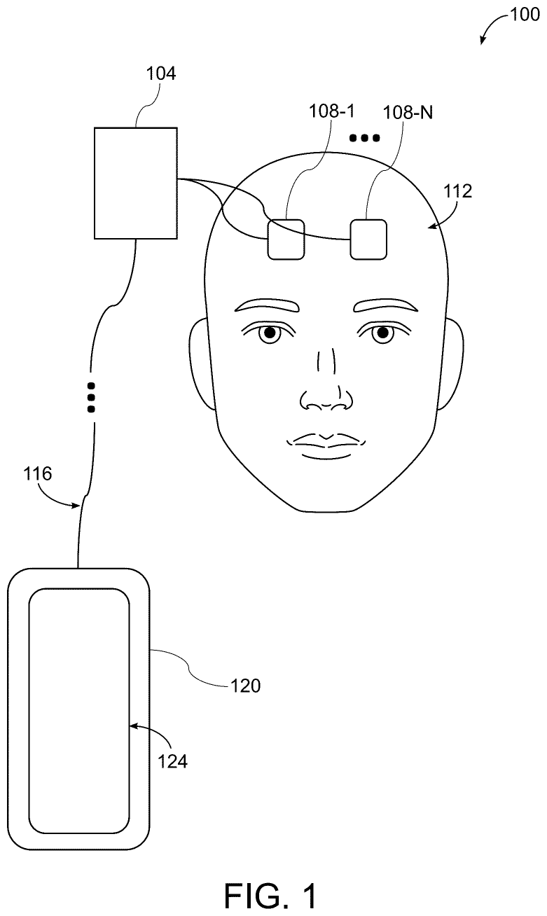

For voltage gradients to remain high enough in working brain circuits when current flows default downwards in aging, resistance levels need to increase. Users may conceive this increase of resistance as a brick wall to be overcome in order to process an item in working memory, wherein bricks can lessen as a lag time is overcome or active processing demands are ongoing. The change in resistance may be imputed by modeling a percentage resistance gain each year, or by assessing a maximum amplitude of voltage from frequency-based representations of time series brain activity measurements (square root), e.g., measurements acquired using the system 100 of FIG. 1, and showing by how much the user could increase blood current flow, e.g. through exercise, and activating brain work to regain some of the loss, for example, with respect to current or voltage. A cardiovascular assessment of pulse output volume may also serve to confirm decreases in fluidic cardiovascular current.

As users may have more trouble relating to a negative concept such as resistance, an additional way to present functioning can include modeling mental processing as a brain current. In mental processing, a current can be thought of as the flow of charge necessary to fire synapses across the series and parallel paths of synaptic clefts relevant to the task. This current and increasing resistance due to aging may be related through threshold activation voltages as above. The original input spectrogram represents the raw electrical signal before decomposing by frequency. If a time associated with a particular task is defined as task_{M}, it can be conceived of as a particular task voltage pulse in time. If the pulse is much shorter than the time constant of an effective capacitor network in a charging step, then it can be characterized as:

$${V}_{c} ( {T}_{pulse} ) = {V}_{pulse} ( 1 - {e}^{- {t}_{p u l s e} / RC} ) .$$

Here, tₚᵤₗₛₑ may correspond to a time of a voltage pulse associated with the particular task and the above formula may relate the capacitive charging described hereinabove with the time of the voltage pulse of the task. When the pulse is so short relative to the capacitor time constant RC, i.e., tₚᵤₗₛₑ<<RC, the charging step may be akin to linear increase in voltage with respect to time. Thus, the capacitor network voltage may be much smaller than the pulse voltage. During the pulse, the current can be considered roughly constant at a value given by:

$${I}_{c} ∼ = {V}_{pulse} / R .$$

Then, the change in processing current from a previous (older in time) sample or time point to the new sample or time point in age progression can be:

$${I}_{{c}_{new}} / {I}_{{c}_{old}} ∼ = {V}_{pulse} / ( {R}_{new} / {R}_{old} ) ,$$

if the pulse voltages are equivalent, for example, for the same task. As the collective R, that is, the resistance at previous and newer time points, may vary by frequency, so does the current I. However, it may be useful to show the decline over time periods sampled.

It can also be instructive to show users why the brain diminishes in processing power as it ages. To that end, repeated cardiovascular pulsation can drive energy delivery via arterial-type walls. Each periodic cardiac outflow can hit the arterial-type wall, stretching the wall barrier if sufficient angular force is delivered, and ensues periodic fluid movement in the brain fluidic milieu on the other side. This movement may not be greater than the pulsatile delivery, as the brain creates no movement of its own. Thus, it may be bounded at maximum to pulsatile delivery's energy transduction. Power is then the rate at which this energy is delivered. Power in an electrical circuit is defined by voltage and current, i.e., Power=V*I, or Power=V²/R. Generally speaking, delivered voltage could be derived from spectrogram data, i.e., V_{spectrogram}. R could be defined in relative terms as above, such that the change in resistance R_{new}/R_{old}) can be utilized. The change in delivered power may be

$${( {V}_{spectrgram} )}^{2} / ( {R}_{new} / {R}_{old} ) or$$

$${( {V}_{spectrogram} )}^{2} * ( {R}_{old} / {R}_{new} ) .$$

As such, as people age, they may be working against relatively decreased delivery of energy which fuels brain processing power. Such a relationship may be shown by embodiments described herein.

It may further be instructive to show users how a task input may be related to task processing. According to an example embodiment, an input is a visual scene, for example, a mathematical calculation on paper to be performed. The brain result can include pulse entrainment in the beta sub-band. A transfer function could capture this relationship, as the brain processes input through electrical signals. Events or operations associated with the task input may include focusing on a region of interest on the paper and extracting legible characters. Measurements of electrical activity associated with such operations may be used to generate a spectrogram and a resulting ascertained voltage can be labelled as V_{task input}. This can be derived after observing beta sub-band pulse entrainment and working backwards in time to identify the most recent visually classified task. Pulse entrainment magnitude can constitute V_{task output}. The transfer function V_{task output}/V_{task input} or such in constituent frequencies could be thought of as a closed system from which human vision perceives some task input, all the way through to mentally executing that same task in working memory. This classic system ratio can be used to understand system behavior under various input conditions in school or training, and understand system stability and instability (wherein instability can relate to an output being undefined or failing to converge on a sub-band center frequency), to better comprehend human transformation from sensory to thought.

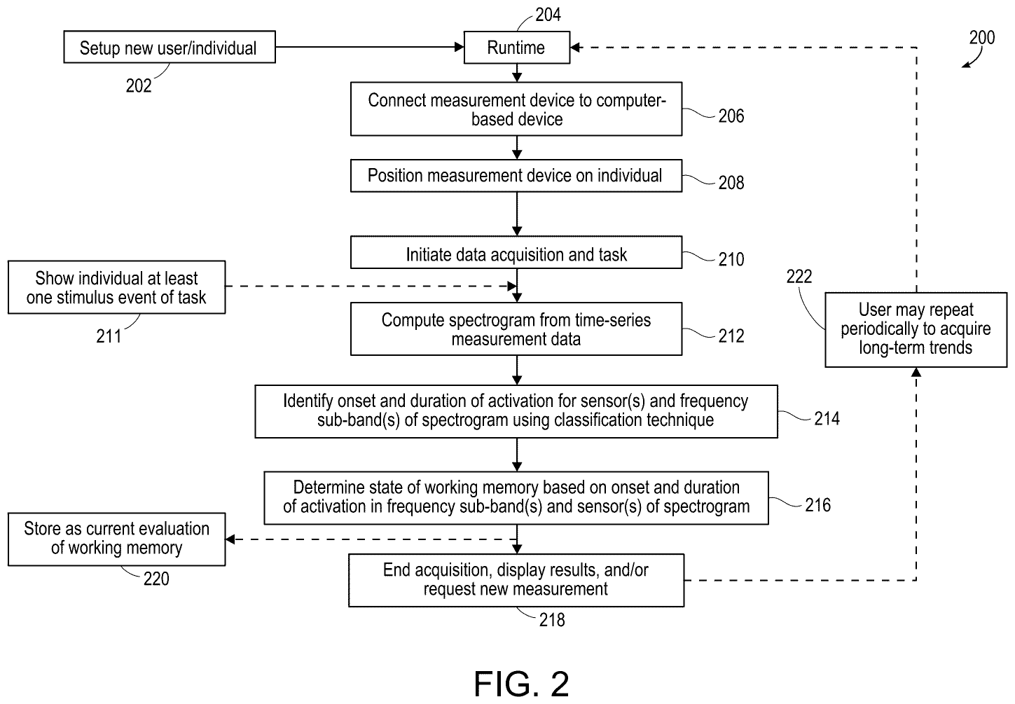

FIG. 1 illustrates an example embodiment of a system for determining a state of working memory of a brain. The system 100 includes a measurement device 104 with at least one sensor 108-1 . . . 108-N. A sensor of the at least one sensor 108-1 . . . 108-N may be positioned on a forehead or other suitable region of a head of an individual 112. The measurement device 104 may be configured to connect 116 to a computer-based device 120 with an electronic screen 124 through a wireless or a wired connection. The computer-based device 120 may record measurement data from the measurement device 104 and the individual may initiate a runtime, receive a task comprising at least one stimulus event, view the results of the runtime, and other features on the electronic screen 124 of the computer-based device 120. The computer-based device 120 may be further configured to obtain temporal information of the at least one stimulus event, compute a spectrogram from the measurement data, identify any number of onsets and any number of durations that may correspond with brain processing, and determine the state of the brain (e.g., a state of working memory) based on the any number of onsets and the any number of durations. Further information regarding the runtime are provided in FIG. 2.

FIG. 2 illustrates a flow diagram of an example embodiment of a method 200 for determining a state of working memory that may utilize at least some of the elements described in FIG. 1. Continuing with reference to FIG. 2, an individual may setup a new user or new user account on a computer-based device at step 202 and initiate a runtime at step 204. A measurement device comprising at least one sensor may be connected with the computer-based device at step 206 and may be positioned on the individual at an anatomical region to be studied at step 208. Measurement data may be collected by the measurement device and recorded by the computer-based device and a task comprising at least one stimulus event may be initiated at step 210. The computer-based device may be configured to record a start time, an end time, or a start time and an end time corresponding to a stimulus event of the at least one stimulus events shown to the individual 211. At step 212, the measurement data collected may be analyzed to compute a spectrogram, the spectrogram comprising for a sensor of the at least one sensor at least one frequency sub-band over the period of time. A classification technique may be used to identify any number of onsets and any number of durations of activation, the activation including bursting or spiking, within the at least one frequency sub-band of the spectrogram at step 214. An onset of the any number of onsets may comprise a time point in the spectrogram with an amplitude exceeding a threshold value and a duration of the any number of durations may comprise a length of time in the spectrogram in which the amplitude remains above the threshold value. At step 216 the state of working memory of the brain may be determined for a point in time of the period of time by the any number of onsets identified and the any number of durations identified for the at least one sub-band of the spectrogram. The current evaluation of the state of working memory may be saved into the memory of the computer-based device at step 220 and the runtime may be concluded at step 218. The runtime may further be repeated in the future at step 222 to acquire long-term trends of the state of working memory.

In some example embodiments for determining the state of working memory, the computer-based device may be a cell phone, a computer, a laptop computer, or a tablet. The sensors of the measurement device may be electrodes, in which case a measurement modality may be electroencephalography. The measurement device may be configured to connect to the computer-based device using a wireless or a wired connection. Optionally, the computer-based device may include a camera that may be used to guide placement of sensors on the anatomical region to be studied. The placement of sensors guided by the camera may use a computer vision method.

In other example embodiments, the task may include one or more stimulus events. The one or more stimulus events may include a word puzzle, a sudoku puzzle, a visual maze, an auditory task, or other cognitive or mental tasks. The task may be presented on the computer-based device, a separate computer-based device, or another form of media, for example, paper, an audio tape, or others. The computer-based device may interface with the other media or use the camera on the computer-based device to track the start time, the end time, or the start time and the end time of a stimulus event of the task.

In certain example embodiments, the measurement data comprises sensor data from the at least one sensor over a period of time, the period time defined by the runtime or the individual. Sensor data is captured in real time per sensor, for example, readings for a given sensor n includes Sₙ[t, voltage difference between two electrodes, amplitude]. The data can be organized as a 2-dimensional array. Either intrinsically or via a Fourier transform, the data can be converted into a PreparedSignal[(f₁, amplitude₁, phase₁), (f₂, amplitude₂, phase₂), . . . (fₙ, amplitudeₙ, phaseₙ)]. Frequencies in a target sub-band such as beta frequencies can be extracted, for example, by applying a band-pass filter after taking the Fourier transform. The band-pass filter may also be applied beforehand to reduce a size of a dataset. Then, an algorithm to detect repeated frequency bursting in a periodic fashion can be applied, where successive passes through the filtered dataset can vary the period amount to capture all possible bursting, i.e., t, 2t, 3t, up to a maximum threshold_{period}. Bursting patterns that do not extend for long enough in threshold time or number of spikes are discarded as entrainment candidates. For each entrainment pattern, an array entrainment [tₛₜₐᵣₜ, frequency, pulse_length] can hold spiking candidates, which may include multiple spiking candidates.

The entrainment array with the first or lowest time can be selected as the topmost task candidate to be processed. With this time in mind, any task start occurring before this time can be evaluated by using the camera trained on a user's work. If such a task start can not be identified in a threshold_{task activation} time proximity, then it can be presumed that the associated bursting is due to a prior task underway. Processing for the first entrainment array candidate can be considered complete and a next entrainment array entry candidate in time then becomes the topmost task candidate to be processed accordingly (or a combined, blended, or convolved beta clock speed could be evaluated).

Retrieval of memories, in other words, replay or decoding from posterior brain regions, can exhibit time lags or positive phase shifts as the waves propagate. When the brain encodes, for example, predictive processing in advance of sensory input, there can be a phase shift forward. In some cases, phase shifts may not be directly obtained from a lone sampling point (for example, from a single electrode) but may be obtained from a system or sensor comprising a plurality of EEG electrodes. When multiple sensors are utilized, then for the same frequency burst pattern, a time shift can be computed into phase derivation. Conversion via frequency-based decomposition can yield both magnitude and phase information. Phase shifts during entrainment can occur in the gamma sub-band, beta sub-band, or another sub-band. Thus, successive encoding, decoding, or both encoding and decoding due to phase coordination can co-occur within a task interval in working memory.

The sensor data may be associated with an index of a sensor of the at least one sensor and may be organized in a form of a matrix, for example, RawData[sₓ,t] in which s represents the index of the sensor and t represents time. For a sensor of the at least one sensor, the data may comprise an array RawData[t]. One or more pre-processing techniques may be applied to the measurement data to generate preprocessed temporal data, PreparedSignal[sₓ,t]. For a sensor, the data may comprise an array, PreparedSignal[t].

A spectrogram may then be computed from the preprocessed temporal data, returning output matrix FourierSignal[s,f] which contains amplitude and phase information of the frequency sub-band at s sensor index and f frequency sub-band. In some embodiments, FourierSignal may comprise one sensor and one point in time, that is, the entire experiment duration, and may be represented by FourierSignal[f]. The spectrogram may be computed using a frequency-based decomposition technique, for example, a discrete Fourier transform (DFT).

Starting and stopping a sensor measurement window at any time, implies that brain signals being measured are likely aperiodic, that is, non-periodic in nature. An aperiodic collective signal can be handled by first contracting a periodic signal that equals the measured, preprocessed signal PreparedSignal[t] over one period. With a periodic signal for which PreparedSignal[t] is one period, as the Fourier transform is integrated over all time values from negative infinity to positive infinity, transform values can only reflect the non-zero signal, that is, the measured signal values.

The discrete Fourier transform yields the constituent frequencies that manifest in a signal in time as they are superposed upon one another in the time domain. A band-pass filter may be applied in the frequency domain to procure a target frequency sub-band to be evaluated. For some embodiments, the target frequency sub-band of the at least one frequency sub-band may be alpha waves, beta waves, gamma waves, or delta waves, or others. In other embodiments, the frequency sub-band of the at least one frequency sub-band may span a portion of the ranges of the alpha waves, beta waves, gamma waves, or delta waves or some combination thereof.

An example embodiment of a bandpass filter for beta waves may comprise BandpassFilter(f)=1 for f≥12 Hz and f≤40 Hz and BandpassFilter(f)=0 elsewhere. Entries in FourierSignal[s,f] may be processed through the bandpass filter on a per-sensor and per-frequency basis. Additional embodiments of the bandpass filter may include frequencies in the gamma wave frequency range, or another sub-band range or some combination thereof.

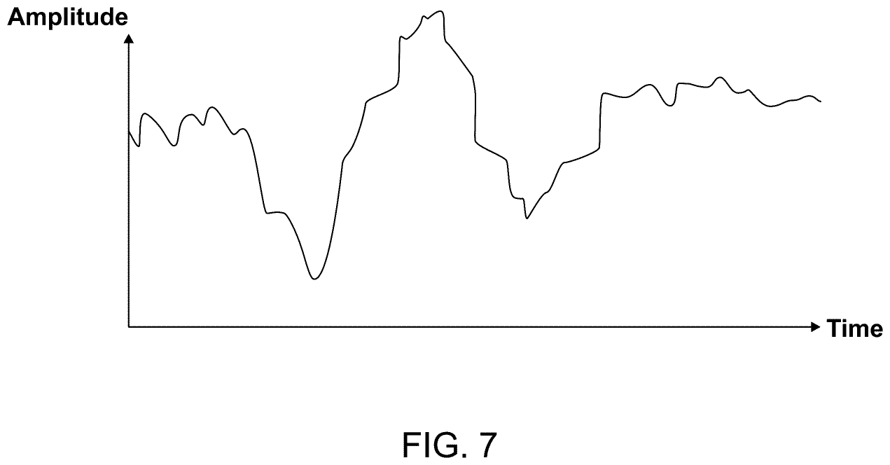

FIG. 7 is a schematic illustration of a plot of time series data of brain activity acquired using a sensor, according to an example embodiment. As described hereinabove, a given sensor may include sensor readings, for example, EEG sensor readings of electrophysiological activity, over time. Furthermore, a system, e.g., the system 100 described herein with reference to FIG. 1, may include one or more sensors. A plot of time series data of brain activity similar to the plot of FIG. 7 can be generated for measurement data of each sensor. As described hereinabove, measurement data can be organized into a matrix RawData[s, t] wherein s represents an index of a sensor and/represents measurement time.

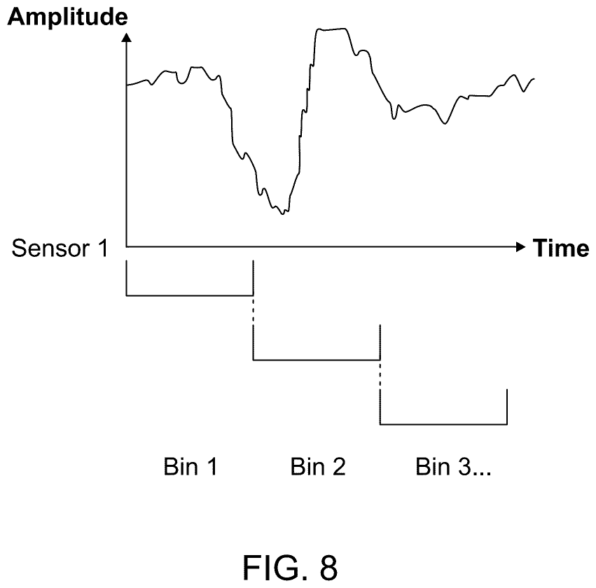

FIG. 8 illustrates dividing time series data, such the time series data of FIG. 7, into discrete time bins, according to an example embodiment. As described herein, spectral information of brain activity can vary over time and progression of such variations may be useful for determining a state of working memory of a brain. This may be achieved by discretizing time series data from a given sensor (e.g., Sensor 1) into time bins (e.g., Bin 1, Bin 2, Bin 3, . . . , Bin n) and determining spectral characteristics of each time bin. A frequency-based decomposition, e.g., Fourier analysis, may be applied to the time series data of each time bin to compute the spectral characteristics. The spectral signal may be represented as FourierSignal[s, f], wherein s represents sensor index and f represents a frequency in a sub-band, as described herein.

Bandpass filters, for example, filters selecting for specific wave ranges such as alpha or beta waves, can be applied to screen out background signals.

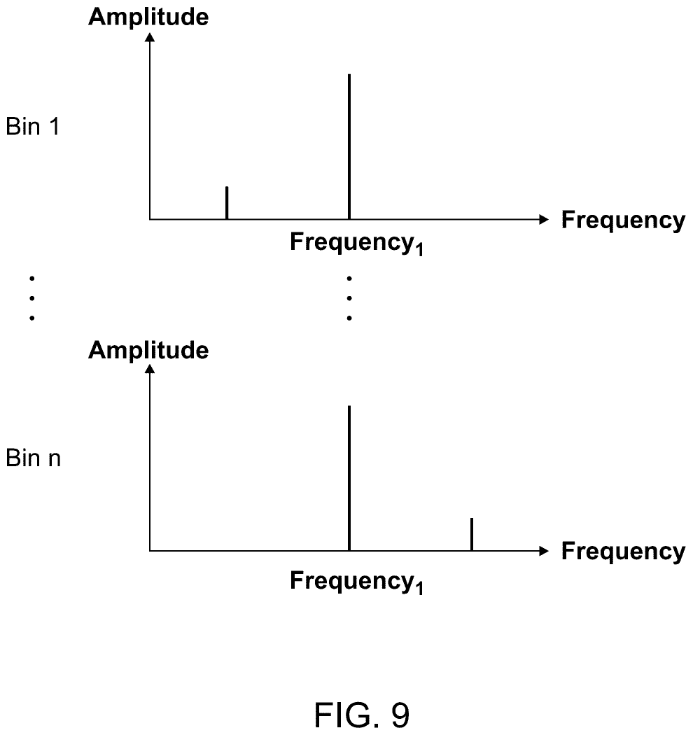

FIG. 9 is a schematic illustration of plots of frequency components of two discrete time bins (e.g., Bin 1 and Bin n). A dataset may comprise n time bins, of which only Bin 1 and Bin n are exemplified. The frequency components of the time bins can be used to identify common frequencies in successive time bins. As a particular example, the frequency components may further be used to identify common frequencies from successive time bins that exceed a threshold that may be indicative of brain activation. Such data may be represented as, according to an example embodiment, CommonFrequencySuccessiveBins [s₁, f₁, bin_{w}, . . . , binₓ] . . . [sₙ, fₙ, bin_{y}, bind], wherein each array entry lists a contiguous range of bin indices wherein frequencies appear for a given source. For a sensor, multiple entrainment frequencies may be present.

The classification technique used for identifying the onset and the duration may include a principal component analysis, a support vector machine, a machine learning method, or other methods. EEG data is complex and full of noise, artifacts, and information. Classification of the onset of an activation and the duration of an activation may take into account prior spiking or bursting, and the sensor of the at least one sensor may be considered as an independent data source as measurement data may vary from one sensor to another. In some embodiments, a recurrent neural network (RNN) may learn from training data and may handle sequential data.

A bidirectional RNN may learn from training data, deal with sequences, and presume outputs depending upon inputs. An array comprising measurement data, e.g., sensor data, bandpass-filtered sensor frequency, time start, and pulse length, can be used as functional input. Doing so may be advantageous by reducing the size of the matrix datasets processed. Bandpass-filtered sensor frequencies could include beta sub-bands, gamma sub-bands, alpha sub-bands, cross sub-bands, or any other sub-band meriting evaluation.

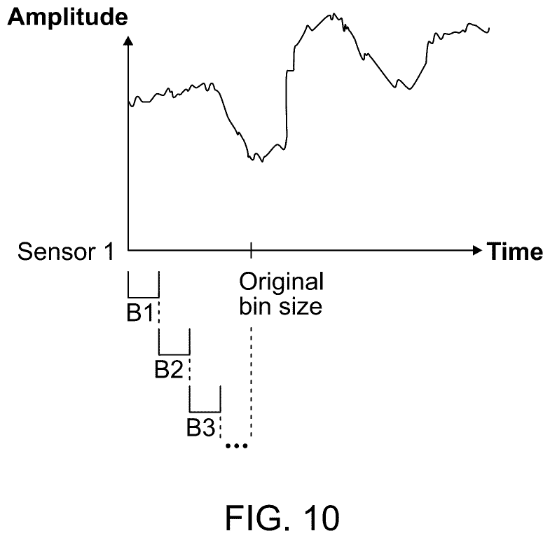

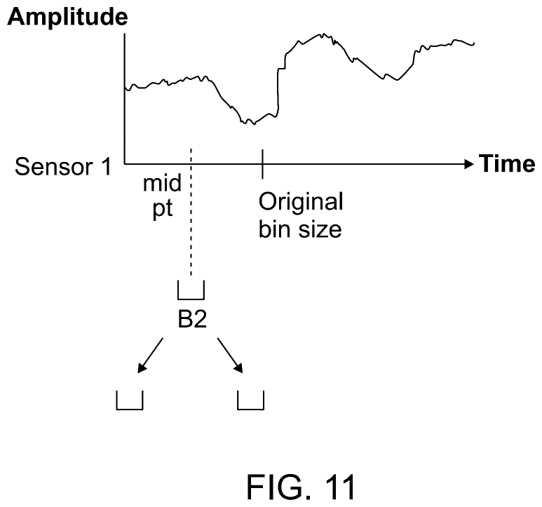

The forward process can include as an input a frequency-based Fourier transform, e.g., a discrete Fourier Transform (DFT), fast Fourier transform (FFT), or short-time Fourier transform (STFT), of a sensor's time series data sliced into time interval bins. As there is no inverse method that can pinpoint a time of start or end, search strategies that optimize a search space may be used to efficiently search the bins for target frequency components. Example methods for performing such a search are described herein with reference to FIGS. 10 and 11. The bins can be compared for co-occurring frequencies.

Bin size can reflect chosen sub-band frequency floors to capture frequencies without being too fine grained, e.g., for beta waves, 12 Hz translates to 1/f=0.083 seconds. According to such an example, a co-occurring frequency can be chosen as a topmost candidate. If the chosen frequency happens to be higher than 12 Hz, the bin size can be accordingly readjusted and reassessed as a higher frequency may re-occur within a smaller unit of time. To find where entrainment begins in a finer-grained time window, a search through the finer-grained time windows may be utilized. To assess in greater specificity, for example, a frequency's start time, the earliest time bin sampled should be further sliced into finer-grained bins and evaluated for presence or absence of the frequency. The forward process can identify associations of impulses, such as impulse entrainment and higher frequency modulation (e.g., a gamma frequency band, and correlate such associations with tasks. The association can occur with one or more sensors of a system, e.g., the system 100 of FIG. 1.

FIG. 10 illustrates an example method of finding a starting time and an ending time for a frequency entrainment evaluated in time series data, such as the time-series data of FIG. 7, obtained from a given sensor (e.g., Sensor 1). The frequency entrainment may be identified, for example, by sequentially searching again through the time series data (e.g., each time bin B₁, B₂, B₃, . . . ). A time associated with the time bin can be marked as including an activity within a specific frequency range (for example, a carrier signal enabling brain activity), and a start and end time of the entrainment may be identified.

FIG. 11 illustrates another example method of finding a starting time and an ending time for a frequency entrainment evaluated in time series date, such as the time-series data of FIG. 7, obtained from a given sensor (e.g., Sensor 1). A more efficient search method may include, as a first step, an initial time bin (e.g., B2). If a frequency component f₁ is identified in the initial time bin, an earlier time bin may be searched in a subsequent step. Alternatively, a subsequent time bin may be searched if the frequency component f₁ is not present within the initial time bin. Such an approach of directional searching may be useful for reducing a search space.

In parallel, backwards processing can work in an opposite direction, by taking resulting entrainment and modulating frequencies with accompanying time(start) and time(end) (or bin index stage if chosen bins do not overlap). Using a time bin previous to time(start), for example, and invoking frequency-decomposition can be useful for ascertaining whether time previous to an identified task start corresponds to absence of such frequencies and, therefore, a correct state of a no impulse entrainment. If no impulse entrainment has been detected in the forward process for a given set of time bins, then the backward process will complete as is without impulse entrainment, and there are no associated start times and end times to assess. Thus, backwards processing can run in parallel with forward processing.

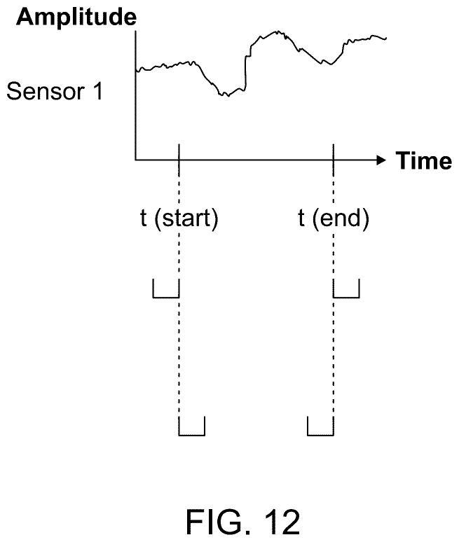

FIG. 12 illustrates an example backwards processing of time series data to identify and verify associated start and end times of a selected entrainment frequency, according to an example embodiment. An entrainment of a given frequency component may be determined to include a start time t(start) and an end time t(end). Time bins can be formed around an identified t(start) and t(end). Frequency decomposition of time before t(start) should confirm a lack of the given frequency component and time after t(end) should similarly confirm the lack of the given frequency component. Discretization of time between t(start) and t(end) into time bins and analysis thereof should further confirm a presence of the given frequency component.

Forward processing can result in a hidden state: a topmost bursting candidate that initiates an identified task, including its pulse length. Backwards processing can work in an opposite direction, by taking a resulting entrainment to find time(start) and time(end), as disclosed hereinabove, and assess whether time previous to a start of task entrainment corresponds to a correct state of no existing tasks and no impulse entrainment. During training, a loss function can help to tune the network but must be chosen so that stability is enhanced without sacrificing optimization. In the frequency domain, to evaluate task start or stop time, the array FourierSignal[s,f] represents all (filtered) frequencies across sensors in each sensor's column. By using an RNN, the data may be processed across multiple time steps, with array entries that retain entrainment higher frequency candidates: frequency, spike level amplitude, time(start) slot, time(end) slot, and sensor number that was present in the prior step. This array may be used in subsequent steps to fit for a correct task.

To identify a likely paired task, a context vector, updated by parallel sensory processing, may be used. Start and end times for each beta or lower frequency entrainment candidate can be compared to a candidate task to determine whether a time interval corresponds thereto. If not, a next entrainment candidate can be processed for the time interval fit. (In parallel, via another backwards process, a next task delineation can also be processed against an entrainment candidate for the time interval fit. Once a successful entrainment candidate is identified, corresponding higher modulating frequencies can be populated by evaluating their start and end times (e.g., higher modulating frequencies that fall within the entrainment start and end interval identified.

To train the RNN, sample time and/or frequency-based data from sensors may be arranged into a matrix, then passed through a filter layer which scans the first row or column data for a sufficient activation pattern. If a sufficient activation pattern is found, the sample data is retained as a layer memory state, then the next row or column is processed through the same layer in sequence. The RNN output is dependent upon information both from the prior memory states and the current layer output. RNN has been used with biological EEG sensor input to process in the past for meaning.

RNNs may be used to process the FourierSignal[s,f] matrix on a per-sensor basis across multiple time steps. In some embodiments, activation functions of the RNN may determine if a sufficient amplitude of a frequency sub-band of the at least one frequency sub-band has been achieved and if sufficient duration of sufficient amplitudes of the frequency sub-band has been achieved to constitute activation. The activation functions may also determine the presence of a single activation or a sequence of activations. The onsets and the durations of the activations for all sub-bands of the at least one frequency sub-band may be stored and may be used to identify a state of working memory, the state including task invocation or task execution.

A separate RNN algorithm may be utilized for evaluating various encoding or decoding task states, with bandpass filtering in the target range. In the frequency domain, to evaluate wave directionality, the array FourierSignal[s,f] represents all (filtered) frequencies in each sensor's row, sampled across time tₙ for each sampled time interval. If a subsequent sensor yields a corresponding same beta frequency (within a threshold), with a time lag, then the specific task may constitute a task involving one or more activations, e.g., encoding via gamma modulating signal from one brain region to another brain region or decoding from other regions.

By using RNN, the data may be processed across multiple time steps, with memory states that retain any sufficient state: band frequency, start time slot, current time slot and sensor number. Sufficient time steps (over threshold) in a beta frequency may constitute a traveling carrier wave for a given task, until the next beta frequency invocation for the next task (plus response time lag).

Task encoding or decoding may constitute states and the sensors may detect directionality via sensor placement relative to time lag of a detected activation at a given frequency. It may be possible that other carrier frequencies circulate simultaneously with a given task carrier frequency, similar to FM radio station, wherein multiple frequencies may be broadcast simultaneously to maximize communication channels with minimal interference.

In embodiments using a measurement device comprising multiple sensors, further activation functions may determine directionality of activations across sensors and across time, the directionality of activations across sensors and time may be used to identify encoding or decoding.

Different kinds of task initiation may exist for different types of tasks. For example, handwriting may exhibit different impulse train properties when compared to cognitive tasks, for example, mental arithmetic, e.g., performing a ‘2+2’. Responding to an incoming on- or off-screen hazard, e.g., a thrown ball, may exhibit different task properties when compared to multiple word choice or reading. Relating a task to a pattern of mental task initiation and duration may be useful, as then an individual's mental processing may be more succinctly observed. Further, such patterns may be somewhat anticipated afterwards to occur as a function of task initiation and type. For example, a given pattern may be used to identify further instances of a given task associated with the given pattern.

According to an example embodiment, to associate a task with a pattern of activation of brain activity, a vector model may be built that would abstract an essence and uniqueness of a task. A device may look through a camera lens at a type of visual task that is to be undertaken. A task stimulus event may include any given task, for example, reading, writing, or calculating, but the task stimulus may not be labelled as such, as the domain may be less pertinent than the mental response to it.

According to an embodiment, both a visual task stimulus event at a given time, e.g., time t, and a next beta-frequency impulse train onset, which may occur at time t+N, wherein N represents an offset in time, may be tracked in a vector model as pulse train onset may be material only to a given task occurring at or after the given time of task initiation. Once the beta-frequency impulse train ceases at time t+M, a task may be considered to be finished (or put on hold). The task time interval may be marked as M−N.

As a particular beta frequency sub-band commenced at time t+N and ceased at time t+M, any gamma-sub-band activation taking place in between the time start and end points may constitute a modulating information signal. Restated, the gamma sub-band activation and a duration of gamma sub-band frequency sequences may represent successive regulation of a beta sub-band task. Thus, between t+N and t+M, each gamma sub-band frequency onset and successive duration interval may be plotted in time to show the sequence of task processing. A new beta frequency sub-band may subsequently indicate a new processing (sub) task or choice, where a new carrier frequency, e.g., the new beta frequency sub-band, may carry gamma modulating frequencies pertaining to that next time interval, i.e., the time interval of the new beta frequency sub-band.

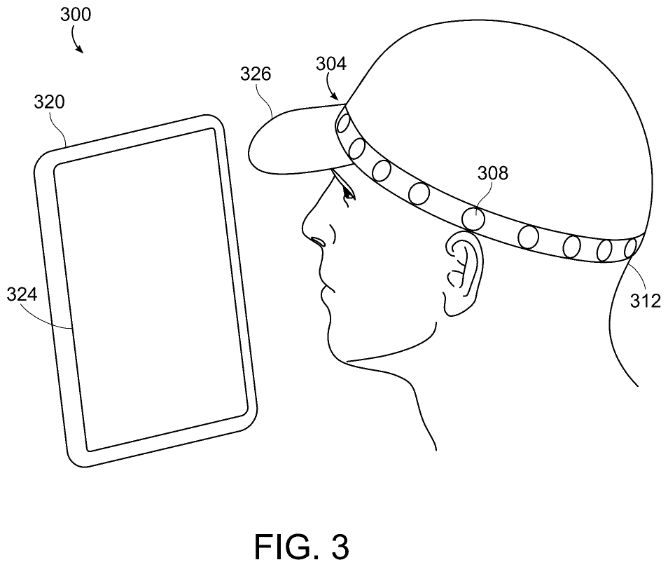

FIG. 3 illustrates an example embodiment of a system 300 for evaluating a state of working memory of a brain with sensors, e.g., the sensor 308, positioned on a head 312 of a user. The system 300 may include a measurement device 304, which includes sensors, including the sensor 308, disposed on an internal surface of a hat, e.g., a cap 326. The cap 326 may be useful for ensuring contact between the sensors 308 and the head 312 of the user. The system further includes a computer-based device 320, e.g., a tablet, which may be communicatively coupled to the measurement device 304. The computer-based device 320 may include an electronic screen 324 and may be used for one or more of controlling data acquisition using the sensors, rendering representations of tasks for the user, rendering representations of measurements acquired using the sensors, or rendering representations of states of working memory.

In an example application of an embodiment of the present invention, the state of working memory may be determined for individuals in a classroom setting. Brain-brain synchrony is described in an article “Measuring Brain Waves in the Classroom” by van Atteveldt et al., Front Young Minds, 2020, 8:96, and in an article titled “Brain-to-Brain Synchrony in the STEM Classroom” by Davidesco, CBE Life Sciences Education, 2020, 19(3), the teachings of which are incorporated by references in their entirety.

For an individual with Attention-Deficit/Hyperactivity Disorder (ADHD), synchronization and attaining a target frequency may be more difficult to achieve. An instructional tool would show a student when they are far off a teacher's center frequency and whether they are closing a gap between the student's center frequency and the teacher's center frequency on a screen. For some kinds of tasks that are well standardized, such as math calculations, alignment of gamma frequency patterns may be useful. Students who wish to improve their task execution could each wear a measurement device. For such a purpose, a teacher could be prerecorded or teaching live with a measuring device. Frequencies may be a “fuzzy” type match, i.e., provide an approximate match, so further processing, for example, by applying frequency thresholds, can be utilized. Visual indicators, audio indicators, or tactile indicators may be used to help present the gap to the individual. This application may be utilized in a classroom setting, the classroom setting being in-person, over telecommunications, or as a recording as non-limiting examples, to assist as few as one participant in attaining a center frequency nearer to the target frequency.

According to some embodiments, a lack of coherent or consistent impulse trains across measured time periods may be present. This lack of pulse trains may indicate a nascent lack of or existence of working memory. In a particular case, the lack of coherent or consistent impulse trains may indicate lack of ability to concentrate. However, working memory may develop, for example, in a toddler, over a course of months or years. Changes in measurable impulse trains may indicate a progression continuum toward working memory skill formation.

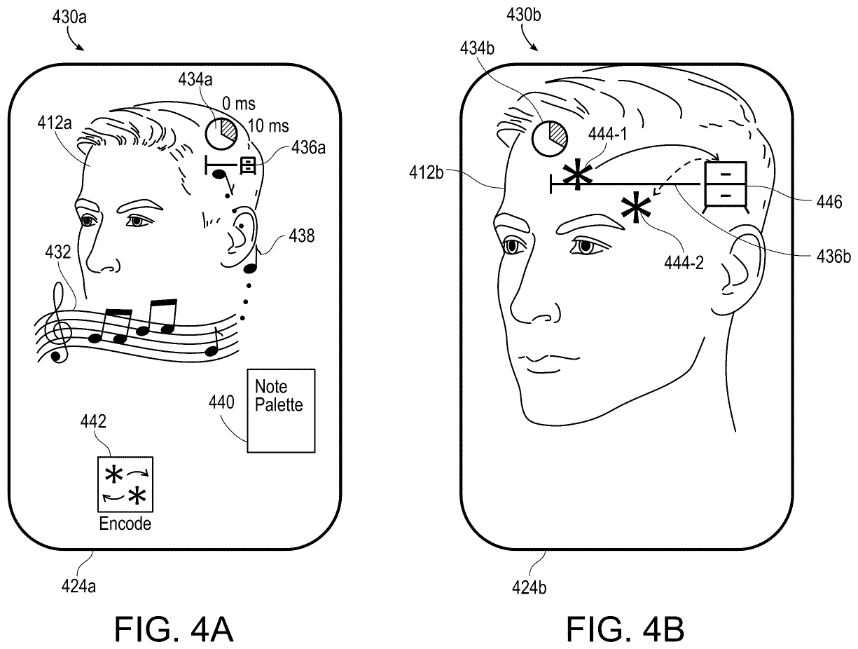

FIGS. 4A and 4B illustrate graphical interfaces of task execution by a user, which may be rendered on a computer-based device (e.g., the device 120, 320 described herein with reference to FIGS. 1 and 3) in a system for determining a state of working memory of the brain of the user, according to an example embodiment. As described herein, a computer-based device may be configured to communicatively couple to a measurement device. The computer-based device may further be used to perform one or more of controlling data acquisition using the measurement device, presenting a task to a user, rendering a representation of a task being performed, or rendering a representation of measurements of working memory of a brain.

FIG. 4A illustrates a graphical interface 430a including a head 412a of a user. The graphical interface 430a may be rendered on an electronic display 424a, which may be similar to the electronic screen 124 described herein with reference to FIG. 1. The user may be wearing a measurement device (not shown, but may be similar to the measurement device 104, 304 described herein with reference to FIGS. 1 and 3) and may be performing a task, e.g., a task involving listening to music, which may be represented by a musical clef 432. The graphical interface 430a may further include a stopwatch 434a, which may indicate a time course or a duration of a measurement associated with working memory of the user. The graphical interface 430a may further include a timeline 436a, which may show ongoing time incrementing and, optionally, when a given tasks starts and when decoding and encoding labeled events of the working memory of the user occur. Decoding would occur when the user is recalling music that was similar or previously the same and encoding could occur when hearing a new musical strain. For musical tasks, a symbol, e.g., a musical note 438, may be used to represent task completion or task execution. Additionally, interactive features may be implemented. For example, to ensure that a user is prepared and focused on a given task, the user may be required to tap the musical note 438, wherein the user may choose a tap length corresponding to a duration of a previously played melody note that next plays. The Note/Palette icon could be used to select a different melody or allow the user to generate a new note sequence.

FIG. 4B illustrates another graphical interface 430b including a head 412b of a user. The graphical interface 430b may be rendered on an electronic display 424b, which may be similar to the electronic screen 124 described herein with reference to FIG. 1. The graphical interface 430b includes a stopwatch 434b, which may indicate a time course or a duration of a measurement of a user-specified task and may include task start, encode-labeled events, or decode-labeled events as non-limiting examples. Encoding events may be represented as storing an item, e.g., a star 444-1, into a file cabinet symbol 446 and decoding events may be represented along a timeline as extracting an item, e.g. a star 444-2, from a file cabinet symbol 446. The encoding events and decoding events may be rendered as they are occurring (with respect to a measurement) or replayed after a measurement. Further interaction with elements of the graphical interface 430b may provide additional information, e.g., interacting with the file cabinet symbol may yield more explicit imagery (e.g., dendritic tree) for memory encoding or decoding.

Systems and methods described here can be used with existing brain activity sensors, for example, EEG sensors. Examples of available brain activity sensors are described below.

BrainBit is a wearable EEG device, for example, a head band, with Bluetooth connection to a computer-based device. Brainbit's EEG Waves app shows brain activity in real time using the BrainBit device. It tracks Alpha, Beta, and Theta waves. Further information is available at the website www.brainbit.com.

The Neurosity Crown is another EEG device for the detection of brain waves. Further information is available at the website www.neurosity.co.

The Callibri is a multifunctional sensor configured to monitor muscle using electromyography, brain activity using EEG, and heartrate using electrocardiogramaditional information is available at www.callibri.com.

Divergence is a neurofeedback software platform capable of supporting a range of EEG devices, including the Neurosity Crown and the Brain Bit, and can be found at www.divergenceneuro.com.

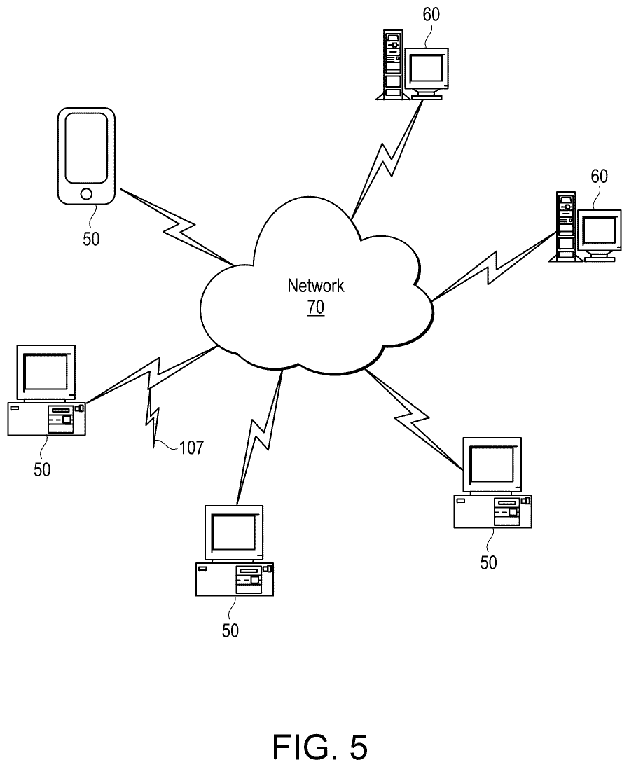

FIG. 5 illustrates a computer network or similar digital processing environment in which the present invention may be implemented.

Client computer(s)/devices 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable.

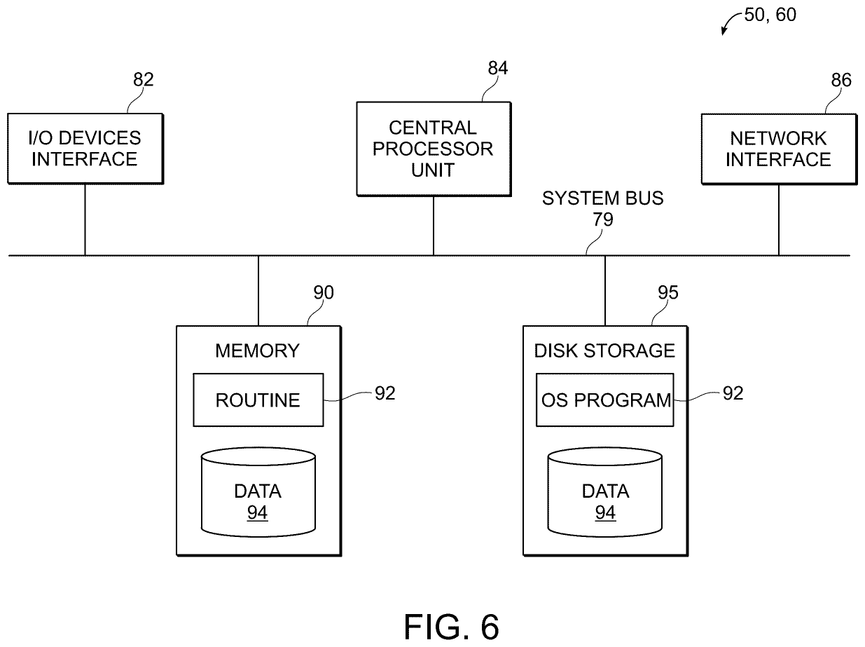

FIG. 6 is a diagram of the internal structure of a computer (e.g., client processor/device 50 or server computers 60) in the computer system of FIG. 5. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 5). Memory 90 provides volatile storage for computer software instructions 92 and data 94 used to implement an embodiment of the present invention (e.g., classification technique, measurements, and supporting code detailed above). Disk storage 95 provides non-volatile storage for computer software instructions 92 and data 94 used to implement an embodiment of the present invention. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

In other embodiments, the program product 92 may be implemented as a so-called Software as a Service (SaaS), or other installation or communication supporting end-users.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method for determining a state of working memory of a mammalian brain, the method comprising:
positioning a measurement device on an individual at an anatomical region to be studied;
obtaining measurement data of the individual's brain from the measurement device, wherein the measurement data comprise time series data collected from at least one sensor of the measurement device over a period of time, the period of time including at least one stimulus event;
obtaining temporal information of a start time, an end time, or both a start time and an end time corresponding to the at least one stimulus event;
computing a spectrogram of the time series data of the measurement data collected from the at least one sensor;
identifying using a classification technique any number of onsets of activation and any number of durations of activation within at least one frequency sub-band of the spectrogram in time, wherein an onset of the any number of onsets comprises a time point in the spectrogram with an amplitude exceeding a threshold value and a duration of the any number of durations comprises a length of time in the spectrogram in which the amplitude remains above the threshold value;
determining a state of working memory of the brain in the period of time based on the any number of onsets identified and the any number of durations identified of the at least one frequency sub-band of the spectrogram; and
outputting the state of working memory, the state of working memory temporally correlated with the at least one stimulus event using the temporal information.

2. The method of claim 1, wherein the measurement data comprise recordings of electrical activity.

3. The method of claim 1, wherein the state of working memory includes task invocation, task execution, memory encoding, or memory decoding.

4. The method of claim 1, wherein computing the spectrogram includes applying a frequency-based decomposition method.

5. The method of claim 4, wherein computing the spectrogram further includes applying one or more data preprocessing techniques to the measurement data.

6. The method of claim 1, wherein the at least one frequency sub-band includes alpha waves, beta waves, gamma waves, theta waves, delta waves, or some combination thereof or therebetween.

7. The method of claim 1, wherein the classification technique includes a machine learning method, a principal component analysis, a support vector machine, a Bayesian inference, a Recurrent Neural Network, or a combination thereof.

8. The method of claim 1, wherein the anatomical region comprises a forehead of the individual.

9. The method of claim 1, wherein the anatomical region includes a pre-frontal cortex or a frontal lobe.

10. The method of claim 1, wherein the at least one stimulus event includes a working memory task, a motor task, or a cognitive task.

11. The method of claim 1, further comprising determining a health status of the working memory of the individual based on the state of working memory and one or more of the any number of onsets of the at least one sub-band, the any number of durations of the at least one sub-band, or the temporal information corresponding to the at least one stimulus event.

12. A system for determining a state of working memory of a mammalian brain, the system comprising:
a measurement device comprising at least one sensor, the measurement device positioned on an individual at an anatomical region to be studied;
a computer-based device configured to connect with the measurement device, the computer-based device further configured to:
obtain measurement data of the individual's brain from the measurement device, wherein the measurement data comprise time series data collected from the at least one sensor over a period of time, the period of time including at least one stimulus event;
obtain temporal information of a start time, an end time, or both a start time and an end time corresponding to the at least one stimulus event;
compute a spectrogram of the time series data of the measurement data collected from the at least one sensor;
identify using a classification technique any number of onsets of activation and any number of durations of activation within at least one frequency sub-band of the spectrogram in time, wherein an onset of the any number of onsets comprises a time point in the spectrogram with an amplitude exceeding a threshold value and a duration of the any number of durations comprises a length of time in the spectrogram in which the amplitude remains above the threshold value;
determine a state of working memory of the brain in the period of time based on the any number of onsets identified and the any number of durations identified of the at least one frequency sub-band of the spectrogram; and
output the state of working memory, the state of working memory temporally correlated with the at least one stimulus event using the temporal information.

13. The system of claim 12, wherein the at least one sensor is configured to measure electrical activity.

14. The system of claim 12, wherein the state of working memory includes task invocation, task execution, memory encoding, or memory decoding.

15. The system of claim 12, wherein the computer-based device is configured to compute the spectrogram by applying a frequency-based decomposition method.

16. The system of claim 15, wherein the computer-based device is further configured to compute the spectrogram by applying one or more pre-processing techniques.

17. The system of claim 12, wherein the at least one frequency sub-band includes alpha waves, beta waves, gamma waves, theta waves, delta waves, or some combination thereof or therebetween.

18. The system of claim 12, wherein the computer-based device is configured to apply as a classification technique a machine learning method, a principal component analysis, a support vector machine, a Bayesian inference, a Recurrent Neural Network or a combination thereof.

19. The system of claim 12, wherein the computer-based device is a cell phone, a tablet, a computer, or a laptop computer.

20. The system of claim 12, wherein the computer-based device is configured to provide the stimulus event via a graphical interface.

21. The system of claim 20, further comprising the graphical interface, wherein the computer-based device is configured to output a representation of the state of working memory via the graphical interface.
